(19) United States
(12) Patent Application Publication
(10) Pub. No.: US 2010/0104622 A1
(43) Pub. Date: Apr. 29, 2010

(54) Ligand Targeted Nanocapsules for the delivery of RNAi and other Agents

(76) Inventor: Remco Alexander Spanjaard, Brookline, MA (US)

Correspondence Address: Maurizio Cattaneo, 196 Samoset Ave, Quincy, MA, 02169, US

(21) Appl. No.: 12/259,211

(22) Filed: Oct. 27, 2008

(51) Int. Cl. A61K 9/127

(52) U.S. Cl. 424/450

(57) ABSTRACT

A carrier system for the delivery of therapeutic and/or diagnostic agents is described. The carrier system is comprised of ligands and a biodegradable polycation for complexing polyanionic molecules such as RNAi, said polycation forming a coating on the outer surface of anionic or neutral liposomes. Also disclosed is a method for using the composition to deliver to target cells and enhance cell membrane penetration of therapeutic and/or diagnostic agents.

### FIELD OF THE INVENTION

This invention relates to carriers and the delivery of therapeutic and/or diagnostic agents which are preferably targeted for site-specific release in cells, tissues and organs. In preferred embodiments, this invention relates to ligand-receptor mediated systems for target cell-specific delivery of nucleic acids, DNA, RNAi, oligonucleotides, proteins, peptides, drugs and/or diagnostic agents into cells.

### BACKGROUND OF THE INVENTION

The present invention relates to carriers for the delivery of therapeutic and/or diagnostic agents which are preferably targeted for site-specific release in cells, tissues or organs. More particularly, this invention relates to ligand-targeted polycation-coated liposomes which comprise a ligand and a biodegradable polycation for complexing polyanionic molecules such as nucleic acids and RNAi.

In spite of a substantial body of research and progress which has been achieved for the development of a system whereby a pharmaceutical agent can be selectively delivered to the site in need of treatment, many pharmaceutical delivery systems for the treatment of various diseases such as cancer, autoimmune, infectious and inflammatory diseases impart substantial risk to the patient.

Cancer continues to take a high toll on the American population (ACS. Facts and Figures. American Cancer Society. 2008) and an increasing number of biological and clinical studies in the last 20 years has been devoted not only to the discovery of mechanisms underlying such genetic disease, but mainly to the identification of specific targets for new molecular therapies. In fact, traditional systemic therapies such as chemo- and hormone-therapy have been showing a) considerable side effects, due to the susceptibility of normal cells to chemotherapy insults and b) failure in killing all cell populations in the context of the tumor, due to the fact that a portion of tumor cells is able to activate generic mechanisms of resistance to chemotherapeutic agents or hormones upon treatment. Also, the ultimate goal would be the realization of a molecular classification of all human cancers, based on which individual tumors could be treated independently of their origin, simply identifying the main alterations specifically responsible for tumor growth.

Although it is desired to concentrate a cytotoxic agent at a targeted site, current cancer treatment protocols for administering these cytotoxic agents typically call for repeated intravenous dosing, with careful monitoring of the patient. The dose is selected to be just below the amount that will produce acute (and sometimes chronic) toxicity that can lead to life-threatening cardiomyopathy, myelotoxicity, hepatic toxicity, or renal toxicity.

Previous attempts to administer such drugs by direct injection into the location of the organ having the malignancy are only partially effective, because of migration of the drug from that location and as a result of extensive tissue necrosis from extravasation. Such dispersion cannot be totally prevented, with the result that excessive quantities of drug need to be administered to attain a desired result.

The direct injection of cytotoxic agents into solid tumors of the breast, bladder, prostate and lung using conventional cytotoxic chemotherapeutic agents as adjuvants to surgery and/or radiotherapy has had limited success in prolonging the lives of patients. This is partially due to the dose limitations imposed by the acute and chronic toxicity to tissues or organ systems beyond those that are targeted.

For a compound to be an effective pharmaceutical agent in vivo, the compound must be readily deliverable to the patient, not rapidly cleared from the body, have a tolerable level of toxicity, and be able to reach the site within the body where it is needed.

In recent years there has been a strong focus on discovering new ways of targeting cancer cells. A major limitation to current cancer therapies is that they harm many healthy cells in the process.

The general problem of drug targeting consists of at least three basic issues. They are the following:
1. How to ensure the most effective interaction of drugs with target cells, including their proper binding on cell membranes and intracellular transport.
2. How to effectively deliver drugs towards certain target cells avoiding unfavorable drug distribution in the organism and their disintegration on their way to the targets.
3. How to avoid nonspecific action of drugs on nontarget cells.

Viral vectors are very effective in terms of transfection efficiency but they have limitations in vivo such as immunogenicity and unintended recombination (Douglas J T and Curiel D T. Targeted gene therapy. Tumor Targeting 1:67-84, 1995; Thomas C E, Ehrhardt A, Kay M A. Progress and problems with the use of viral vectors for gene therapy. Nat Rev Genet 4, 346-358, 2003; Verma I M and Somia N. Gene-therapy—promises, problems and prospects. Nat Med 389:239-242, 1997).

Non-viral delivery systems include cationic liposomes and cationic polymers. Cationic constructs are an attractive choice since simple mixing with negatively charged DNA or RNA in vitro leads to electrostatically-driven self-assembly into polyelectrolyte complexes (Kabanov A V and Alakhov V Y. New approaches to targeting bioactive compounds. J Cont Release 28:15-35, 1994).

Liposomes are artificial single, oligo or multilamellar vesicles having an aqueous core and being formed from amphipathic molecules. The cargo may be trapped in the core of the liposome or disposed in the membrane layer or at the membrane surface. Today, liposomal vectors are the most important group of the nonviral delivery systems. More specifically, cationic liposomes or lipids have been used widely in animal trials and/or clinical studies. Cationic liposomes have being used to deliver oligonucleotides and siRNA (Semple S C, Klimuk; Sandra K, Harasym T, Hope M J, Ansell S M, Cullis P, Scherrer P, Debeyer Dan. Lipid-encapsulated polyanionic nucleic acid, U.S. Pat. No. 6,858,225, 2005; Wheeler, J J, Bally M B, Zhang Y P, Reimer D L, Hope M, Cullis P R, Scherrer P. Lipid-nucleic acid particles prepared via a hydrophobic lipid-nucleic acid complex intermediate and use for gene transfer, U.S. Pat. No. 5,976,567, 1999; Wheeler, J J, Hope M, Cullis P R, Bally M B. Methods for encapsulating plasmids in lipid bilayers U.S. Pat. No. 6,815,432, 2004). Also, a great deal of effort has been made over the years to develop liposomes that have targeting vectors such as monoclonal antibodies (mAbs) attached to the bilayer surface (Barbet J, Machy P and Leserman L O. Monoclonal antibody covalently coupled to liposomes: specific targeting to cells. J. Supramolec Struct Cell Biochem 16:243-258, 1993; Jones M N and Hudson M J. The targeting of immunoliposomes to tumor cells (A431) and the effects of encapsulated methotrexate. Biochim Biophys Acta 1152:231-242, 1993; Torchilin V P. Immobilization of specific proteins on liposome surface: systems for drug targeting. In: Liposome Technology vol. 3, CRC, Boca Raton, Fla., pp. 29-40, 1993).

Although cationic systems provide high loading efficiencies, they lack colloidal stability, in particular after contact with body fluids. Ionic interactions with proteins and/or other biopolymers lead to in situ aggregate formation with the extracellular matrix or with cell surfaces. In addition, cationic liposomes, although they provide effective synthetic transfection systems, their use in vivo is limited by general toxicity, complement activation and liver and lung tropism (Dass C R. J. Pharm. Pharmacol 54:593-601, 2002; Dass C R. Lipoplex-mediated delivery of nucleic acids: factors affecting in vivo transfection. J Mol Med 82:579-91, 2004; Filion M C, Phillips N C. Toxicity and immunomodulatory activity of liposomal vectors formulated with cationic lipids toward immune effector cells. Biochem Biophys Acta 1329:345-356, 1997; Hirko A, Tang F, Hughes J A. Cationic lipid vectors for plasmid delivery. Curr. Med. Chem 10:1185-1193, 2003; Lv H, Zhang S, Wang B, Cui S, Yan J. Toxicity of cationic lipids and cationic polymers in gene delivery. J Cont Release 114:100-9, 2006; Ma Z, Li J, He F, Wilson A, Pitt B, Li S. Cationic lipids enhance siRNA-mediated interferon response in mice. Biochem Biophys Res Comm 330:755-9, 2005; Romoren K, Thu B J, Bols N C, Evensen O. Transfection efficiency and cytotoxicity of cationic liposomes in salmonid cell lines of hepatocyte and macrophage origin. Biochem Biophys Acta 1663:127-34, 2004; Zhang J-S. Liu F, Huang L. Implications of pharmacokinetic behavior of lipoplex for its inflammatory toxicity. Adv Drug Del Rev 57:689-98, 2005). In addition, it has been shown that antibodies become immunogenic when coupled to these liposomes (Phillips N C and Dahman J. Immunogenicity of immunoliposomes: reactivity against species-specific IgG and liposomal phospholipids. Immunol Lett 45:149-52, 1995).

Cationic polymers have also frequently been selected for use as non-viral vectors. Studies with linear polycations such as poly(lysine) and a ligand such as a receptor-recognizing molecule do mimic some basic functions of natural viruses (Kabanov A V, Yu V, Alakhov V Y, Chekhonin V P. Enhancement of macromolecular penetration into cells and nontraditional drug delivery systems, In: Skulachev V P (Ed.) Sov. Sci. Rev., D., Physicochem. Biol., Harwood Academic Publishers, New York, Vol. 11, part 2, pp. 1-75, 1992, Kabanov A V and Kabanov V A. DNA Complexes with Polycations for the Delivery of Genetic Material into Cells. Bioconj Chem 6:7-20, 1995). On the basis of this work Trubetskoy et al. (Trubetskoy V S, Torchilin V P, Kennel S J and Huang L. Use of N-terminal modified poly(L-lysine)-antibody conjugate as a carrier for targeted gene delivery in mouse lung endothelial cells. Bioconjugate Chem 3:323-327, 1992) developed a system using poly (L-lysine) antibody conjugate as a carrier for targeted gene delivery in mouse lung endothelial cells. However, the cationic polymers most often used, including poly(lysine), polyethyleneimine and PAMAM dendrimers (Wu G Y, Wu C H. Evidence for targeted gene delivery to HEP G2 hepatoma cells in vitro. Biochem 27:887-892, 1998) are very toxic to cells. Although the process of complexation with DNA or RNA, with the consequent charge neutralization counteracts this toxicity, it is nonetheless a concern when one considers the ultimate fate of the construct and the possibility for localized delivery of the polycation, hence the need for non-toxic polycations. Therefore, the toxicity of cationic lipids and polymers is still an obstacle to the application of non-viral vectors to gene therapy.

Recent data shows that a natural cationic biopolymer consisting of a low molecular weight highly purified chitosan was neither toxic nor hemolytic and could be administered intravenously without liver accumulation (Cattaneo M V and Demierre M F. Biodegradable Chitosan for Topical Delivery of Retinoic Acid. Drug Del Tech 1:44-48, 2001; Richardson S C W, Kolbe H V J and R Duncan. Potential of low molecular mass chitosan as a DNA delivery system: biocompatibility, body distribution and ability to complex and protect DNA. Int J Pharm 178:231-243, 1999). The lactate form of this biocompatible polymer also showed rapid blood clearance and excellent DNA complexation resulting in inhibition of DNA degradation by DNase II, and greater DNA interaction than poly-L-lysine (Richardson S C W, Kolbe H V J and Duncan R, 1999, Weecharangsan W. Opanasopit P. Ngawhirunpat T, Rojanarata T, Apirakaramwong A. Chitosan lactate as a nonviral gene delivery vector in COS-1 cells. AAPS Pharm Sci Tech 7:66, 2006). Several investigators have included a moiety such as PEG to increase stealth and the circulation time of the drug carrier. PEG micelles and liposomes have been prepared according to a method described in Zalipsky et al. (Polyethylene glycol chemistry, Biotechnical and Biomedical Applications (J. M. Harris Ed.) Plenum Press, pp. 347-370, 1992). In addition, since chitosan shows high affinity for lipids, several investigators have utilized chitosan derivatives as coating materials for liposomes (Guo J, Ping Q, Jiang G, Huang L and Tong Y. Chitosan-coated liposomes: characterization and interaction with leoprolide. Int J Pharm 260:167-173, 2003; Janes K A, Calvo P, Alonso M J. Polysaccharide colloidal particles as delivery systems for macromolecules. Adv Drug Deliv Rev 47:83-97, 2001; Takeuchi H., Yamamoto H. and Kawashima Y. Mucoadhesive nanoparticulate systems for peptide drug delivery. Adv Drug Deliv Rev 47:39-54, 2001).

Transmembrane transport of nutrient molecules is a critical cellular function. Because practitioners have recognized the importance of transmembrane transport to many areas of medical and biological science, including drug therapy, peptide therapy and gene transfer, there have been significant research efforts directed to the understanding and application of such processes. Thus, for example, transmembrane delivery of nucleic acids has been encouraged through the use of protein carriers, antibody carriers, liposomal delivery systems, electroporation, direct injection, cell fusion, vital carriers, osmotic shock, and calcium-phosphate mediated transformation. However, many of those techniques are limited both by the types of cells in which transmembrane transport is enabled and by the conditions of use for successful transmembrane transport of exogenous molecular species. Further, many of these known techniques are limited in the type and size of exogenous molecule that can be transported across a membrane without loss of bioactivity.

One method for transmembrane delivery of exogenous molecules having a wide applicability is based on the mechanism of receptor-mediated endocytotic activity (REA). Unlike many other methods, REA can be used successfully both in vivo and in vitro. REA involves the movement of ligands bound to membrane receptors into the interior of an area bounded by the membrane through invagination of the membrane. The process is initiated or activated by the binding of a receptor-specific ligand to the receptor. Many REA systems have been characterized, including those recognizing peptide growth factors such as epidermal growth factor (EGF) and insulin growth factor, (IGF), galactose, mannose, mannose 6-phosphate, transferrin, asialoglycoprotein, transcobalamin (Vitamin B.sub.12), alpha-2-macroglobulins, insulin.

REA has been utilized for delivering exogenous molecules such as proteins and nucleic acids to cells. Generally, a specific ligand is chemically conjugated by covalent, ionic or hydrogen bonding to an exogenous molecule of interest (i.e. the exogenous compound), forming a conjugate molecule having a moiety (the ligand portion) that is still recognized in the conjugate by a target receptor. Using this technique the hepatocyte-specific receptor for galactose terminal asialoglycoproteins has been utilized for the hepatocyte-specific transmembrane delivery of asialoorosomucoid-poly-L-lysine non-covalently complexed to a DNA plasmid (Wu, G. Y. J. Biol Chem. 262:4429-4432, 1987); the cell receptor for epidermal growth factor has been utilized to deliver polynucleotides covalently linked to EGF to the cell interior (Myers A E, A method for internalizing nucleic acids into eukaryotic cells; European Patent Application No. 86810614, 1988).

With respect to RNAi delivery, a major limitation to the use of RNAi in vivo is the effective delivery of RNAi to the target cells (Behlke M A. Progress towards in vivo use of siRNAs. Mol Ther 13:644-70, 2006; Dykxhoorn D M, Lieberman J. Knocking down disease with siRNAs. Cell 126:231-5, 2006). As a general rule, a molecule such as RNAi faces not one but a combination of problems. Although RNAi is a potentially useful therapeutic approach to silence the targeted gene of a particular disease, its use is limited by its stability in vivo. In particular, RNAi faces the problem of penetration into cells while avoiding disintegration in body fluids and intracellularly. Therefore, despite some progress achieved in this field, no reliable tool for siRNA targeting has yet been developed.

RNAi that specifically interferes with gene expression at the transcriptional or translational levels have the potential to be used as therapeutic agents to control the synthesis of deleterious proteins associated with viral, neoplastic or other diseases. These treatment strategies have been shown to block the expression of a gene or to produce a needed protein in cell culture. However, a major problem with these promising treatments, is adapting them for use in vivo.

Recently, there has also been an important focus on the application of RNAi to silence specific genes involved in cancer proliferation. Although RNAi has shown great efficacy in the selective inhibition of gene expression, the therapeutic applications of RNAi is currently limited by their low physiological stability, slow cellular uptake, and lack of tissue specificity.

Thus, there exists a need for a drug delivery system which can be utilized for the delivery of RNAi, which may also include therapeutic and/or diagnostic agents. There is also a need for a drug delivery system which can be used for site-specific release of RNAi, therapeutic and/or diagnostic agent in the cells, tissues, or organs in which a therapeutical effect is desired to be effected.

### BRIEF SUMMARY OF THE INVENTION

The present invention relates to carriers for the delivery of therapeutic and/or diagnostic agents which are preferably targeted for site-specific release in cells, tissues or organs. More particularly, this invention relates to ligand-targeted nanocapsules which comprise a ligand and a nanocapsule containing a polycation for complexing polyanionic molecules such as nucleic acid and RNA. Different coatings, ligands, RNAi, pharmaceuticals and/or diagnostics can be used tailored (customized) to the intended target cell in order to achieve maximum antitumor activity of the system as shown in FIG. 1.

The combination of a low-toxicity biodegradable polycation with anionic or neutral liposomes, said polycation being coupled to target-specific ligands, produces a carrier system with a low potential systemic toxicity. The polycationic coating makes this system exquisitely suitable for coupling polyanionic agents such as siRNA. In addition, the liposomal component of the carrier system can be used to entrap therapeutic and/or diagnostic agents. Further modifications of the coating by molecules such as Polyethylene glycol (PEG) can be implemented to further increase the blood circulation time. The polycation coating serves as a platform for both complexing the polyanions as well as covalently binding of ligands that increase the identification and subsequent penetration of the target cell membrane.

The cationic biopolymer acts as a transfecting agent and a carrier for anionic macromolecules as well as a matrix for coupling of different ligands results in a dramatic (at least 2 log order) increase in the transfection (delivery) efficiency of the nanocapsules compared to an antibody control particle (normal IgG). Furthermore, the liposomal component acts as a carrier for other therapeutic and/or diagnostic agents, thus separating the polyanionic agents such as RNAi from other therapeutic or diagnostic agents that are entrapped in the liposome. This would allow simultaneous delivery of agents that can interfere with two different biochemical pathways in the target cell.

This invention reveals that transfection efficiency without the specific antibody is negligible. Thus, it is conceivable that our ligand-coupled nanocapsules will achieve high tumor-specific delivery and reduce toxicity.

Ligand-targeted nanoparticles are interesting vectors since they may help protect the encapsulated drug from in vivo degradation as well as minimize the drug's toxicity as a result of the targeting feature of the molecular entity. In this context, the term “ligand” refers to a biomolecule which can bind to a specific receptor protein located on the surface of the target cell or in its nucleus or cytosol. The ligand is internalized through a process termed receptor-mediated endocytotic activity, where the receptor binds the ligand, the surrounding membrane closes off from the cell surface, and the internalized material then passes through the vesicular membrane into the cytoplasm. The ligand then becomes the transfecting agent.

In one embodiment, the ligand may be an antibody, hormone, pheromone, or neurotransmitter, or any biomolecule capable of acting like a ligand, which binds to the receptor. When the ligand binds to a particular cell receptor, endocytosis is stimulated. Examples of ligands which have been used with various cell types to enhance biomolecule transport are galactose, transferrin, the glycoprotein asialoorosomucoid, epidermal growth factor, fibroblast growth factor and folic acid.

If the ligand is chemically coupled to a carrier which contains or is complexed to an anionic macromolecule, the macromolecule can then enter the cytoplasm. The carrier can be a cationic polymer which will further enhance cell membrane penetration as well as complexing an anionc molecule such as RNAi.

Each carrier system may complex RNAi as well as carry a therapeutic and/or diagnostic agent by entrapment into the liposomal compartment of the carrier.

### BRIEF DESCRIPTION OF THE DRAWINGS

FIG. 1A schematic representation of the carrier system.

FIG. 2(A) Antibody-coupled nanocapsule transfection efficiency to melanoma cells. The quantities used for this experiment were 2, 12.5, 25, 50, and 100 mg. (B) The quantities of antibody-coupled nanocapsules used for this experiment were 10, 15, 20, 25, and 30 mg.

FIG. 3 Nanocapsule transfection efficiency to melanoma cells. The quantities used for this experiment were 20 mg of antibody-coupled nanocapsules containing 3′ Alexa Fluor 488-labeled validated BRAF siRNA. The experiment was repeated twice for the EGFR-mAb nanocapsules. EGFR coupling to A375 melanoma cells is shown in green (FITC label) and siRNA incorporation in A375 melanoma cells is shown in red (Alexa Fluor 488 label). No FITC fluorescence is detectable with the IgG-pAb nanocapsules containing FITC label.

FIG. 4 MTS assay to determine cell viability and proliferation of DOX-encapsulated EGFR-mAb at 2 different dosing (EGFR-H=1.6 ml, and EFGR-L=2.5 ml,) without and with DOX (EGFR+DOX-L and EGFR-DOX-H) after incubation with A375 melanoma cells. The cells were incubated with nanocapsules for 1 hour (Left) and 3 hours (Right), and the cells were removed and incubated in fresh media for a total of 48 hours before assessing cell viability and proliferation. (Error bars represent ±1% standard deviations from the mean).

### DETAILED DESCRIPTION OF THE INVENTION

The present invention relates to carriers for the delivery of therapeutics and/or diagnostic agents which are preferably targeted for site-specific release in cells, tissues or organs. More particularly, this invention relates to ligand-targeted nanocapsules consisting of biodegradable polycation coated liposomes, said polycation intended for complexing polyanionic molecules such as nucleic acid and RNAi and a ligand covalently attached to the polycation for triggering receptor mediated endocytosis.

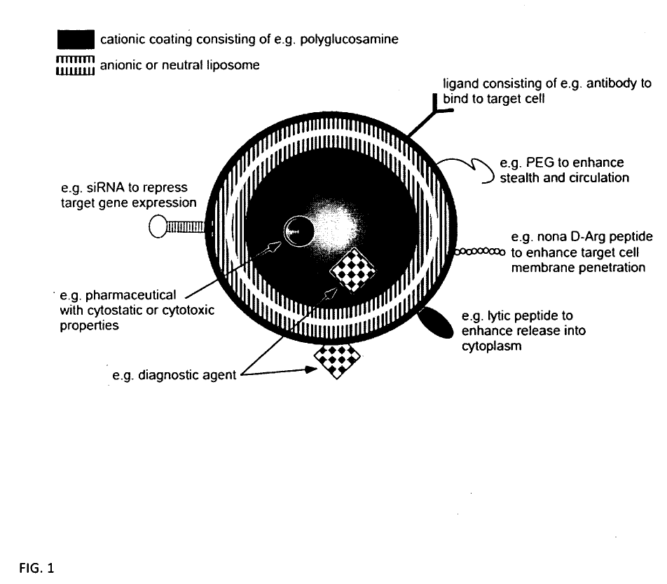

The invention includes, in one embodiment, a nanocapsule including a coating of a hydrophilic cationic polymer on a liposome surface as shown in FIG. 1, the cationic polymer serving to complex polyanionic macromolecules for transport in the circulation and across a membrane of a cell, and the liposome serving to prevent any aggregate formation generally occurring between the cationic polymer coating and the polyanionic macromolecule.

This invention includes in another embodiment the molecular weight of the polycationic polymer which is to be selected for optimal delivery of the polyanionic macromolecule to the target cell. The molecular weight can be in the range from about 100 to about 20,000, Preferably, the molecular weight of the polycationic polymer can be in the range from about 200 to about 10,000. Most preferably, a polycationic polymer with a molecular weight in the range from about 400 to about 2,000 can be used to deliver the polyanionic macromolecule to the cell.

The coating on the liposomal particle may consist of low molecular weight chitosan. The term chitosan refers to a family of polymers having a high percentage of glucosamine (normally 80%-99%) and N-acetylated glucosamine (1%-20%). Low molecular weight chitosan forms a linear polysaccharide chain of molecular weight up to 20,000 Dalton. Chitosan is derived from chitin. It is normally extracted from the exoskeleton of shellfish, mushrooms, or algae and has been previously been described having controlled release properties. Highly purified chitosan, as obtained commercially under the tradename Protasan™ (Novamatrix™, FMC Biopolymers, Philadelphia, Pa.) is both biocompatible as well as biodegradable.

As used herein biocompatible refers to a substance that has limited immunogenic and allergenic ability. Biocompatible also means that the substances does not cause significant undesired physiological reactions. A biocompatible substance may be biodegradable. As used herein biodegradable means that a substance can chemically or enzymatically break down or degrade within the body. A biodegradable substance may form non-toxic components when it is broken down. Moreover, the biocompatible substance can be biologically neutral, meaning that it lacks specific binding properties or biorecognition properties.

Liposomes are artificial single, oligo or multilamellar vesicles having an aqueous core and being formed from amphipathic molecules. The drug or diagnostic cargo may be trapped in the core of the liposome or disposed in the membrane layer or at the membrane surface. Today, liposomal vectors are the most important group of the non-viral delivery systems.

Because our carrier system consists of neutral or anionic coated liposomes which may contain therapeutics and/or diagnostic agents, following intravenous administration, such liposomal vehicles have been found to have a prolonged systemic circulation time. This prolonged circulation time is due to their small size and hydrophilic coating which may minimize uptake by the mononuclear phagocyte system and to their high molecular weight which prevents renal excretion. Liposome-incorporated drugs may accumulate in tumors to a greater extent than the free drug and show reduced distribution into untargeted areas such as the heart. Accumulation of liposomes in malignant or inflamed tissues may be due to increased vascular permeability and impaired lymphatic drainage. The tumor vessels are more leaky and less permselective than normal vessels. Several in vivo studies have shown that liposomes are able to improve the efficiency of anticancer drugs against leukemia and solid tumors.

PEG has many qualities that make it a desirable biocompatible ligand linked as part of the carrier of this invention. First, PEG is commercially available in a variety of molecular masses at low dispersity (Mw/Mn<1.1). It has been shown that PEG2000 will mask lipid-linked antibodies to a lesser degree than PEG5000 (MW of 5000 Dalton) (Mori A et al., Influence of the Steric Barrier Activity of Amphipathic Poly(ethyleneglycol) and Ganglioside GM1 on the Circulation Time of Liposomes and on the Target Binding of Immunoliposomes In Vivo, FEBS Lett. 284(2), 263-266, 1991). The studies indicated that PEG does not exhibit specific affinity for any organ and that its accumulation in tumor tissue is mainly governed by the level of hyperpermeable tumor vasculature (enhanced permeability retention (EPR) effect), irrespective of the molecular mass of the polymer and the tumor loading site. The EPR effect is considered as a passive targeting method, but drug targeting could be further increased by binding to ligands such as antibodies. Targeted liposomes can serve for the delivery of drug to tumors, inflamed tissues or endosomal compartments.

The carrier molecule may also include at least one lysis agent connected to the biodegradable cationic polymer coating. The lysis agent can be selected to break down a biological membrane such as a cell, endosomal, or nuclear membrane, thereby allowing the polyanionic macromolecule to be released into the cytoplasm or nucleus of the cell. As a result of the presence of the lysis agent, the membrane undergoes lysis, fusion, or both. Lysis agents also include viral peptides and synthetic compounds that can break down a biological membrane. A lytic peptide is a chemical grouping which penetrates a membrane such that the structural organization and integrity of the membrane is lost. As an example of a pH-sensitive endosomal lytic peptide is GLFEALLELLESLWELLLEA or GLFEALEELWEAK((e-G-dipalmitoyl) (MacLaughlin F C, Mumper R J, Wang J, Tagliaferri J M, Gill I, Hinchcliffe M, Rolland A P.
Chitosan and depolymerized chitosan oligomers as condensing carriers for in vivo plasmid delivery. J Cont Release 56:259-272, 1998).

The lysis agent may also be covalently linked to the cationic polymer coating by a linker. Such linkers can be 1-Ethyl-3-[3-dimethylaminopropyl]carbodiimide hydrochloride (EDAC) and N-hydroxysuccinimide (NHS), a PEG fragment, a polypeptide, a linear polymer containing an ester bond or an amide bond or a disulfide bond. The linkers are preferably biodegradable linkers.

The carrier molecule may also include at least one targeting moiety connected to the biodegradable polycation coating. The targeting moiety can be selected to bind to a specific biological substance or site herein referred to as the receptor. Thus, the targeting moiety can be chosen based on its ability to bind to a receptor molecule expressed in a specific cell type or specific tissue allowing the polyanionic therapeutic agent to be selectively delivered to the cell or tissue. A targeting moiety refers to those moieties that bind to a specific biological substance or site. The biological substance or site is considered the target of the targeting moiety that binds to it. Ligands are one type of targeting moiety. Ligands have a selective (or specific) affinity for another substance known as the receptor. Because the ligand has a specific affinity for the receptor, the ligand binds to the receptor selectively over other molecules. This selective binding allows for the selective delivery of the polyanionic therapeutic to the target cell. Examples of ligands suitable for targeting cells are an antigen, a hapten, a vitamin, a protein, a polypeptide, biotin, nucleic acids, DNA, RNAi, aptamers, polynucleic acids, a polysaccharide, a carbohydrate, a lectin, a lipid and combination thereof, an antibody, Fab or a fragment thereof.

The receptor for a ligand is an important consideration in selecting a ligand to target a cell. The receptor functions as a type of biorecognition molecule that selectivley binds to the ligand. A receptor can be a protein such as an antibody or a non-protein binding body. As used herein an antibody refers to all classes of antibodies including monoclonal antibodies, chimeric antibodies, Fab fractions, and derivatives thereof.

The carrier of the present invention may also contain covalently nona-D-arginine with or without a spacer as indicated below to the polycation coating to improve target cell penetration.

The invention also provides a method of transporting a polyanionic molecule across the biological barriers of the cell. The cell can be a cell derived from an organism such as hepatocytes, liver cells, kidney cells, brain cells, bone marrow cells, nerve cells, heart cells, spleen cells, stem cells and co-cultures of the above. Moreover, the cells may be from established cell lines such as those available from the American Type Culture Collection (ATCC).

The method of delivering the polyanionic macromolecule to the cell includes complexing the polyanionic macromolecule to the carrier system of the present invention to create a complex. The carrier complex may enter the cell by endocytocis and then escape from the vesicles to gain access to the cytoplasm of the cell. If the target cell is within a cell culture in vitro, the cell can be contacted with the complexed carrier system. If the target cell is within an organism in vivo, the complex may be administered by injecting a solution containing the complex into the circulatory system of the organism. A carrier molecule with a targeting moiety attached will allow the complex to be directed to a target cell with a target corresponding to the targeting moiety. The polyanionic molecule/carrier complex may be administered to an organism by intramuscular, intraperitoneal, intraabdominal, subcutaneous, intravenous, and intraarterial delivery. Other methods of administration of the complex include parenteral, topical, transdermal, transmucosal, inhaled, and insertion into a body cavity such as by ocular, vaginal, buccal, transurethral, rectal, nasal, oral, pulmonary, and aural administration.

The polyanionic molecule can be selected from a number of macromolecules that are useful in the treatment of disease or in laboratory experimentation. In certain configurations of the complex, the polyanionic macromolecule is a nucleic acid such as RNAi, siRNA, DNA, or a combination or derivative thereof. The nucleic acid can be, for example, genomic DNA, plasmid DNA, synthetic DNA, or RNA. Other types of nucleic acids that can be used with the carrier molecule of present invention are, for example, an antisense oligonucleotide, ribozyme, DNAzyme, chimeric RNA/DNA oligonucleotide, phosphorothioate oligonucleotide, 2′-O-methyl oligonucleotides, DNA-PNA conjugates, DNA-morpholino-DNA conjugates, and combinations thereof.

### EXAMPLES

### Example 1

Delivery of RNAi (such as siRNA, shRNA, miRNA)

In this preliminary experiment we discovered that polysaccharide nanocapsules having an antibody such as EGFR covalently attached on the surface can substantially increase the nanocapsule affinity for the target compared to the non targeted counterparts. The nanocapsules directed against the receptor can efficiently bind to and become internalized by cancer cells, resulting in targeted intracellular drug delivery. These targeted nanocapsules efficiently bind to and become internalized by cancer cells in vitro, resulting in targeted intracellular drug delivery of siRNA.

While the principle of antibody-conjugates to target cancer cells has been around for some time, in melanoma this strategy poses an additional problem due to the scarcity of suitable cell surface targets that are required for our specific system. Melanoma markers are generally comprised of 4 types (adapted from Medic S, Pearce R L, Heenan P J and Ziman M. Molecular markers of circulating melanoma cells. Pigment Cell Res 20; 80-91, 2006):
1. Markers involved in melanogenesis. While most specific for the melanocytic lineage, TYR, MITF, PAX3, TRP-1, TRP-2, and gp100 are not expressed on the cell surface and cannot be targeted with antibodies.
2. Melanoma-associated antigens such as are MART-1/Melan-A, p97, GalNAc-T, MIA and MUC18/MCAM are fairly specific however they are either also expressed on many other normal tissues or they are T cell antigens.
3. Tumor-associated antigens are more highly expressed in melanoma, such as cell adhesion molecules, angiogenesis factors, MAGE-A3, S100b, YKL-40, CRP and CRT-MAA. However, these markers too are not specific enough for our purposes or not expressed on the cell surface.
4. Finally, there are markers associated with tumor cell growth, proliferation and migration. Examples are VEGFR, NF-kB, ATF-2, FOS, JUN, MK167, TOP2A, BIRC5, STK6. However, these factors are either not expressed on the cell surface, or not specific enough to be used to target melanoma.

However, there are two cell surface markers, EGFR and TROY, that we believe are excellent candidates for targeting by our antibody-coupled nanocapsules.

### 1. EGFR.

EGFR (ErbB-1) is member of the epidermal growth factor family that includes 3 other members (ErbB-2-4). EGFR is a type 1 receptor tyrosine kinase that is involved in processes related to cellular differentiation and proliferation. It has been well-established that its dysregulation, either via activating mutations or increased expression, contributes to several types of cancers (Woodburn J R. The epidermal growth factor receptor and its inhibition in cancer therapy. Pharmacol Ther 82:241-50, 1999). Apart from epithelial cancers, EGFR is expressed in melanocytic lesions (Ellis D L, King L E, Nanney L B. Increased epidermal growth factor receptors in melanocytic lesions. J Am Acad Dermatol 27:539-46, 1992; Sparrow L E, Heenan P J. Differential expression of epidermal growth factor receptor in melanocytic tumors demonstrated by immunohistochemistry and mRNA in situ hybridization. Australas J Dermatol, 40:19-24, 1999) and a correlation between EGFR expression and tumor progression was noted. Indeed, genetic studies showed amplification of the EGFR gene in a number of cases of melanoma. Consistent with these studies, amplification was more commonly observed in metastatic tumors than early-stage disease (Bastian B C, LeBoit P E, Hamm H, Brocker E B, Pinkel D. Chromosomal gains and losses in primary cutaneous melanomas detected by comparative genomic hybridization. Cancer Res 58:2170-5, 1998; Slovak M L, Persons D, Collins J M, Zhang F, Sosman J A, Tcheurekdjian L. Targeting multiple genetic aberrations in isolated tumor cells by spectral fluorescence in situ hybridization. Cancer Detect Prev 26:171-9, 2002; Udart M, Utikal J, Krahn G M, Peter R U. Chromosome 7 aneusomy: a marker for metastatic melanoma? Expression of epidermal growth factor receptor gene and chromosome 7 aneusomy in nevi, primary malignant melanomas and metastases. Neoplasia 3:245-54, 2001). Because of these findings, EGFR has become a popular clinical target. One therapeutic approach is the development of small molecules such as gefitinib (Iressa), which inhibit its kinase activity. The other strategy is by using monoclonal antibodies that interfere with ligand binding, such as cetuximab.

Even though clinical trials in melanoma with EGFR inhibitors have met with disappointment for reasons that are not yet fully understood (Sosman J A, Puzanov I. Molecular Targets in Melanoma from Angiogenesis to Apoptosis. Clin Cancer Res 12:2376s-2383s, 2006), EGFR expression in melanoma has been considered specific enough to be targeted with EGFR-targeting molecular tools in clinical trials. Based on these studies we decided to use an anti-EGFR antibody that is known to be internalized as one of our antibodies to be conjugated to our nanocapsules. As we will show in our preliminary studies, this strategy is highly promising for the EGFR-dependent delivery of both siRNA and chemotherapeutic drugs into melanoma cells.

### 2. TROY

Recently discovered TROY is a novel, highly specific melanoma-associated type I transmembrane receptor member of the TNF receptor superfamily (TNFRSF) that is aberrantly re-expressed in melanoma (Spanjaard R A, Whren K M, Graves C, Bhawan J. Tumor necrosis factor receptor superfamily member TROY is a novel melanoma biomarker and potential therapeutic target. Int J Cancer 120:1304-10, 2007). TNFRSF members comprise a very large family who, on a macroscopic scale, are important for organizing permanent multicellular structures such as lymphoid organs, hair follicles, sweat glands and bone but also transient structures and activities such as the lactating mammary gland and wound healing (Locksley R M, Killeen N, Lenardo M J. The TNF and TNF receptor superfamilies: integrating mammalian biology. Cell 104:487-501, 2001). TNFRSF members are directly coupled to signaling pathways for cell proliferation and differentiation, and are well-studied with respect to their function in acute immune responses such as inflammation. The other major activity is induction of apoptosis (de Thonel A, Eriksson J E. Regulation of death receptors—Relevance in cancer therapies. Toxicol Appl Pharmacol 207(2 Suppl):123-32, 2005).

TROY is a relatively underexplored molecule, although some aspects have been described. During mouse embryogenesis, TROY is widely expressed (Eby M T, Jasmin A, Kumar A, Sharma K, Chaudhary P M. TAJ, a novel member of the tumor necrosis factor receptor family, activates the c-Jun N-terminal kinase pathway and mediates caspase-independent cell death. J Biol Chem 275:15336-42, 2000; Hisaoka T, Morikawa Y, Kitamura T, Senba E. Expression of a member of tumor necrosis factor receptor superfamily, TROY, in the developing olfactory system. Glia 45:313-24, 2004; Kojima T, Morikawa Y, Copeland N G, Gilbert D J, Jenkins N A, Senba E, Kitamura T. TROY, a newly identified member of the tumor necrosis factor receptor superfamily, exhibits a homology with Edar and is expressed in embryonic skin and hair follicles. J Biol Chem 275:20742-7, 2000; Ohazama A, Courtney J M, Tucker A S, Naito A, Tanaka S, Inoue J, Sharpe P T. Traf6 is essential for murine tooth cusp morphogenesis. Dev Dyn 229:131-5, 2004; Pispa J, Mikkola M L, Mustonen T, Thesleff I. Ectodysplasin, Edar and TNFRSF19 are expressed in complementary and overlapping patterns during mouse embryogenesis. Gene Expr Patterns 3:675-9, 2003), but it is particularly highly-expressed in neuroepithelial cells where it may function to regulate cell proliferation or maintenance of the undifferentiated state (Hisaoka T, Morikawa Y, Kitamura T, Senba E. Expression of a member of tumor necrosis factor receptor superfamily, TROY, in the developing mouse brain. Brain Res Dev Brain Res 143:105-9, 2003). However, after birth expression becomes highly restricted to hair follicles and neuron-like cells in parts of the brain (Hisaoka T, Morikawa Y, Kitamura T, Senba E. Expression of a member of tumor necrosis factor receptor superfamily, TROY, in the developing olfactory system. Glia 45:313-24, 2004; Hu S, Tamada K, Ni J, Vincenz C, Chen L. Characterization of TNFRSF19, a novel member of the tumor necrosis factor receptor superfamily. Genomics 62:103-7, 1999; Ohazama A, Courtney J M, Tucker A S, Naito A, Tanaka S, Inoue J, Sharpe P T. Traf6 is essential for murine tooth cusp morphogenesis. Dev Dyn 229:131-5, 2004; Park J B, Yiu G, Kaneko S, Wang J, Chang J, He X L, Garcia K C, He Z. A TNF receptor family member, TROY, is a coreceptor with Nogo receptor in mediating the inhibitory activity of myelin inhibitors. Neuron 45:345-51, 2005; Pispa J, Mikkola M L, Mustonen T, Thesleff I. Ectodysplasin, Edar and TNFRSF19 are expressed in complementary and overlapping patterns during mouse embryogenesis. Gene Expr Patterns 3:675-9, 2003; Shao Z, Browning J L, Lee X, Scott M L, Shulga-Morskaya S, Allaire N, Thill G, Levesque M, Sah D, McCoy J M, Murray B, Jung V, Pepinsky R B, Mi S. TAJ/TROY, an orphan TNF receptor family member, binds Nogo-66 receptor 1 and regulates axonal regeneration. Neuron 45:353-9, 2005) and perhaps prostate (Eby M T, Jasmin A, Kumar A, Sharma K, Chaudhary P M. TAJ, a novel member of the tumor necrosis factor receptor family, activates the c-Jun N-terminal kinase pathway and mediates caspase-independent cell death. J Biol Chem 275:15336-42, 2000).

The ligand for TROY remains to be established but does not appear to be a known TNFRSF-activating ligand (Mandemakers W J, Barres B A. Axon regeneration: it's getting crowded at the gates of TROY. Curr Biol 15:R302-5, 2005). Recently a function for TROY in normal brain was established when it was found that it is a novel Nogo-66 receptor coreceptor that mediates inhibition of axonal regeneration by myelin inhibitors in the central nervous system (Park J B, Yiu G, Kaneko S, Wang J, Chang J, He X L, Garcia K C, He Z. A TNF receptor family member, TROY, is a coreceptor with Nogo receptor in mediating the inhibitory activity of myelin inhibitors. Neuron 45:345-51, 2005; Shao Z, Browning J L, Lee X, Scott M L, Shulga-Morskaya S, Allaire N, Thill G, Levesque M, Sah D, McCoy J M, Murray B, Jung V, Pepinsky R B, Mi S. TAJ/TROY, an orphan TNF receptor family member, binds Nogo-66 receptor 1 and regulates axonal regeneration. Neuron 45:353-9, 2005).

Regardless of its function, TROY presents an exceptional melanoma-specific membrane protein that can be targeted by specific antibodies against its extracellular domain. It is likely that TROY is also internalized which may increase transfection efficiency, and siRNA and drug delivery to the tumor cell (Schütze S, Tchikov V, Schneider-Brachert W. Regulation of TNFR1 and CD95 signalling by receptor compartmentalization. Nat Rev Mol Cell Biol. 9:655-62, 2008).

Selecting a Therapeutic siRNA to Inhibit Melanoma Cell Proliferation

The next issue is the selection of a suitable therapeutic siRNA to incorporate in our antibody-coupled nanocapsules that can effectively block melanoma cell proliferation. An excellent target for siRNA (as well as kinase inhibitors) is BRAF. There are 3 RAF genes (ARAF, BRAF and RAF1) that encode kinases that serve as down-stream effectors of RAS, but BRAF is particularly important for the development of melanoma. 70% of melanomas contain activating mutations in BRAF (Davies H, Bignell G R, Cox C, Stephens P, Edkins S, Clegg S, Teague J, Woffendin H, Garnett M J, Bottomley W, Davis N, Dicks E, Ewing R, Floyd Y, Gray K, Hall S, Hawes R, Hughes J, Kosmidou V, Menzies A, Mould C, Parker A, Stevens C, Watt S, Hooper S, Wilson R, Jayatilake H, Gusterson B A, Cooper C, Shipley J, Hargrave D, Pritchard-Jones K, Maitland N, Chenevix-Trench G, Riggins G J, Bigner D D, Palmieri G, Cossu A, Flanagan A, Nicholson A, Ho J W, Leung S Y, Yuen S T, Weber B L, Seigler H F, Darrow T L, Paterson H, Marais R, Marshall C J, Wooster R, Stratton M R, Futreal P A. Mutations of the BRAF gene in human cancer. Nature 417:949-54, 2002; Mercer K E, Pritchard C A. Raf proteins and cancer: BRAF is identified as a mutational target. Biochim Biophys Acta 1653:25-40, 2003) which are also present in premalignant atypical or dysplastic nevi (Yazdi A S, Palmedo G, Flaig M J, Puchta U, Reckwerth A, Rütten A, Mentzel T, Hügel H, Hantschke M, Schmid-Wendtner M H, Kutzner H, Sander C A. Mutations of the BRAF gene in benign and malignant melanocytic lesions. J Invest Dermatol 121:1160-2, 2003). Almost 90% of BRAF mutations are of the V600E variety leading to constitutive kinase activation (Wan P T, Garnett M J, Roe S M, Lee S, Niculescu-Duvaz D, Good V M, Jones C M, Marshall C J, Springer C J, Barford D, Marais R; Cancer Genome Project. Mechanism of activation of the RAF-ERK signaling pathway by oncogenic mutations of B-RAF. Cell 116:855-67, 2004; Wellbrock C, Karasarides M, Marais R. The RAF proteins take centre stage. Nat Rev Mol Cell Biol 5: 875-85, 2004) and uncontrolled stimulation of cell proliferation.

Another feature that makes BRAF attractive for our studies is that RNAi approaches have already shown that knock down of BRAF results in reduced tumor growth in both cellular and xenograft animal models (Hoeflich K P, Gray D C, Eby M T, Tien J Y, Wong L, Bower J, Gogineni A, Zha J, Cole M J, Stern H M, Murray L J, Davis D P, Seshagiri S. Maintenance in Melanoma Models. Cancer Res 66: 999-1006, 2006). Note that validated siRNAs against BRAF are commercially available. Thus, siRNA directed against BRAF is our selected method.

### Nanocapsule Preparation

Phospholipon 90 G (phosphatidyl Choline) was obtained from Lipoid (American Lecithin Co., New Jersey), Cholesterol was obtained from Barnet Inc. (Englewood Cliffs, N.J.), Protosan UPB 80/20 (High Purity Chitosan, Molecular weight, 20,000 Dalton) was obtained from NovaMatrix (FMC Biopolymer, Philadelphia, Pa.). Protosan is a highly purified form of chitosan, characterized by a prevalence of amino groups on the □-D-glucose backbone which are available for covalent attachment to protein and peptide targeting agents. The normal mouse IgG and the EFGR (R-1) mouse monoclonal IgG_{2b} were obtained from Santa Cruz Biotechnology, the EDAC, NHS, Ethanolamine Hydrochloride and FITC were obtained from Sigma-Aldrich (St Louis, Mo.).

Liposomes were prepared using the lipid-film method. Multilamellar liposomes (MLL), composed of high purity phosphatidylcholine (PC), and cholesterol (Chol) at a molar ratio of 3:1 (PC:Chol) were prepared by a lipid-film method (Szoka F and Paphadjopoulos D. Comparative Properties and methods of preparation of lipid vesicles (liposomes). Ann Rev Biophys Bioeng 9:467-508, 1980). The lipid concentration in this initial suspension was 60 μmol/ml. To prepare the lipid film 3 grams of Phospholipon 90 G (Phosphatidyl Choline) and 0.5 grams of cholesterol were dissolved in 10 ml of chloroform. The solution was evaporated overnight and then vacuum evaporated for 1 hour to remove any chloroform residue remaining in the film. The lipid film was then rehydrated with 10 ml of PBS and sonicated for 15 minutes. The sonicated liposomes were then coated with the biopolymer to form nanocapsules as described (Takeuchi H, Yamamoto H, Niwa T, Hino T, Kawashima Y. Enteral absorption of insulin in rats from mucoadhesive chitosan-coated liposomes. Pharm Res 13:896-901, 1996). A 0.5% solution of protosan was obtained by dissolving 0.5 grams of Protosan in 100 ml of water with 0.36 grams of lactic acid. 0.5 ml of protosan and 0.5 ml of liposomes were then stirred at room temperature for 1 hour.

### IgG and EGFR Coupling Solutions

The control IgG antibody solution was prepared by stirring 500 μl of 200 μg/0.5 ml normal mouse polyclonal IgG (IgG-pAb), 200 μl of 400 mmol EDAC, and 200 μl of 100 mmol NHS at room temperature for 1 hour, according to a modification of the covalent EDAC/NHS amine crosslinking method (Endoh H., Suzuki Y. and Hashimoto Y. Antibody coating of liposomes with 1-Ethyl-3-(3-Dimethyl-Aminopropyl)Carbodiimide and the effect on target specificity. J Immun Meth 44:79-85, 1981). Similarly, the EGFR antibody solution was prepared by stirring 330 ml of 200 mg/ml EGFR (R-1):sc-101 mouse monoclonal IgG_{2b} (EGFR-mAb), 200 μl of 400 mmol EDAC, and 200 ml of 100 mmol NHS at room temperature for 1 hour.

### IgG-pAb and EGFR-mAb Nanocapsules

To prepare the control IgG-pAb nanocapsules we added 50 μl of the nanocapsules to the coupling IgG solution and stirred at room temperature for 150 minutes. We then added 10 μl of FITC (50 mg/ml) and incubated at room temperature for 1 hour with occasional shaking. We then added 20 μl of 1 M Ethanolamine HCl to the mixture. The excess FITC was removed by repetitive washing of the nanocapsules in PBS. The mixture was then split in 200 μl aliquots and frozen at −20° C. for 2 hours. The frozen aliquots were then lyophilized for 48 hours. Similarly, to prepare the EGFR-mAb nanocapsules we added 70 μl of the nanocapsules to the EGFR solution and stirred at room temperature for 150 minutes. We then added 10 ml of FITC and incubated at room temperature for 1 hour with occasional shaking. We then added 20 μl of 1 M Ethanolamine HCl to the mixture. The mixture was then split into 200 μl aliquots and frozen at −20° C. for 2 hours. The frozen aliquots were then lyophilized for 48 hours.

Complexation of Validated siRNA

5 nM of 3′Alexa Fluor 488-labeled validated BRAF siRNA (QIAGEN Inc., Valencia, Calif.) was dissolved in 200 mg of DEPC-treated water, and then used to rehydrate the lyophilized mAb-coupled nanocapsules aliquots for 30 min at RT. The entrapment procedure was performed immediately before use.

### Cells and Reagents

A375 human melanoma cells, which harbor an activated mutant form of BRAF (V600E), and have high EGFR expression, were obtained from the ATCC and cultured under standard conditions (Demary, K, Wong L, Spanjaard R A. Effects of retinoic acid and sodium butyrate on gene expression, histone acetylation and inhibition of proliferation of melanoma cells. Cancer Lett 163:103-7, 2001). DOX was obtained from Sigma (St. Louis, Mo.) and diluted to a 100 mg/ml stock solution just before addition to the nanocapsules.

### Transfections and Fluorescence Microscopy

A375 cells were seeded in 24 well-plates and grown until 50-70% confluent. Nanocapsules rehydrated in DEPC-treated water were added in different doses and left o/n. The next day, media was removed and replaced by fresh media, and transfection efficiency (# fluorescent cells/# total number of cells counted by bright field) was determined by fluorescence microscopy using an Olympus IX 51 inverted microscope with phase contrast, and fluorescence capabilities coupled to a digital imaging system. FITC was detected via a #41001 filter, Alexa Fluor 488 was analyzed via a #41002 filter (Chroma technology Corp., Rockingham, Vt.). Fluorescence was monitored and detected for up to 7 days after transfection for each experiment.

### Cell Proliferation (MTS) Assay

To assess the effects of DOX-loading of EGFR-coupled nanocapsules on growth of A375 cells, cells were seeded in quadruplicate in a 96-well plate at 7,500 cells/well in a volume of 100 μl/well. The next day, cells were treated with EGFR-mAb nanocapsules±DOX at two different doses: 1.6 or 2.5 mg in 10 μl media for 1 or 3 hr resp. before media with particles was removed and again replaced by 100 μl regular media/well. After 2 days, viable cell numbers were determined by CellTiter 96 Aqueous One Solution Cell Proliferation Assay lit (Promega, Madison, Wis.) which measures bioreduction of MTS into a soluble formazan in viable cells which can be determined in a microplate reader at 490 nM.

### Results

Two sets of experiments were performed with the IgG-pAb and EGFR-mAb nanocapsules. The lyophilized nanocapsules were rehydrated in 200 ml of DEPC treated water 30 minutes before use. They were added in different quantities to 1 ml wells containing A375 melanoma cells and incubated for appr. 24 hours before observation by fluorescence microscopy which allowed quantitative assessment of transfection efficiency.

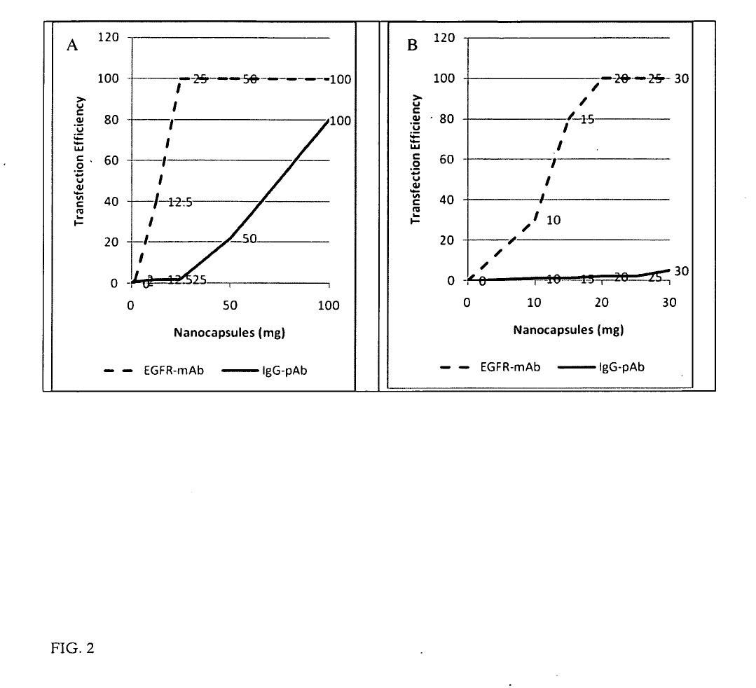

The results in FIG. 2, which show the excellent reproducibility of our system, clearly demonstrate that the presence of the EGFR-mAb on the nanocapsule dramatically increases the transfection (delivery) efficiency of the nanocapsules compared to the IgG-mAb control nanocapsules. At 20 mg, 100% of cells are engaged by the EGFR-coupled nanocapsules whereas IgG control nanocapsules are essentially completely ineffective. We estimate that the difference in transfection efficiency mediated by the EGFR-mAb is at least 10-fold. Thus, the coupling of a monoclonal, melanoma cell surface protein-targeting antibody to our nanocapsules greatly increases affinity for the intended cancer cell. These results imply that our system will be highly suitable for delivery of anticancer agents such as siRNA, and chemotherapeutic drugs. These questions were addressed in the following experiments.

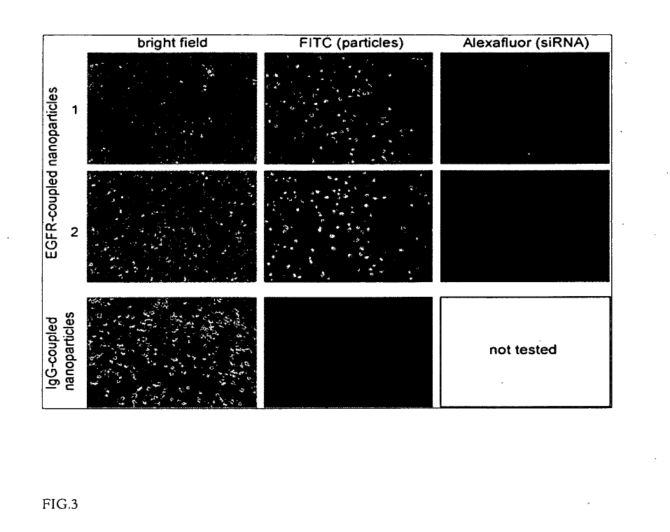

First, we loaded FITC-labeled nanocapsules with a validated AlexaFluor 488-labeled siRNA directed against BRAF, and repeated the above described experiments. As shown in FIG. 3, at 20 mg, again 100% of A375 cells are transfected with the EGFR-antibody-coupled nanocapsules because the bright field and FITC-filter obtained images completely overlap. Interestingly, we find that the same overlap exists with the images detecting the BRAF siRNA. In contrast, no FITC-derived fluorescence is detectable in the IgG-coupled control nanocapsules. Thus, our EGFR-mAb nanocapsules appear to be an excellent, highly specific delivery system for the therapeutic BRAF siRNA.

The delivery of the carrier to the target cell is virtually fully ligand-dependent. In addition, in terms of stability of the complex between RNA and the cationic polymer, we could still detect RNA at least 7 days after transfection in vitro.

It also deserves mentioning that very little cell death was observed even at the highest doses after several days of treatment, although proliferation was mildly inhibited after prolonged incubation. The low-grade cytotoxicity of our nanocapsules, when combined with its longevity in tissue culture, which was confirmed on several other non-related epithelial cell types in these tissue culture experiments (not shown), is an extremely important characteristic for our nanocapsules being clinically suitable delivery agents. These aspects will be further investigated in in vivo studies.

### Example 2

### Delivery of Chemotherapeutic Agent

siRNA holds great promise as it allows specific functional knock-down of critical genes that drive tumor growth and/or survival. However, at the same time, these antibody-conjugated nanocapsules may also be exceptionally suited to deliver extreme localized (because to cancer cells only) chemotherapeutics. The most frequently given drug for advanced stage melanoma is Dacarbazine (DAC). Unlike other chemotherapeutics such as Doxorubicin (DOX), which is essentially ineffective, DAC has produced response rates in the 10-20% range and in rare cases complete remissions have been observed in melanoma patients. Generally, these responses do not result in increased survival and only provide temporary results (McLoughlin J M, Zager J S, Sondak V K, Berk L B. Treatment Options for Limited or Symptomatic Metastatic Melanoma. Cancer Control 15:239, 2008). It is conceivable that targeted delivery of chemotherapeutics with our nanocapsules is much more efficacious than systemic delivery.

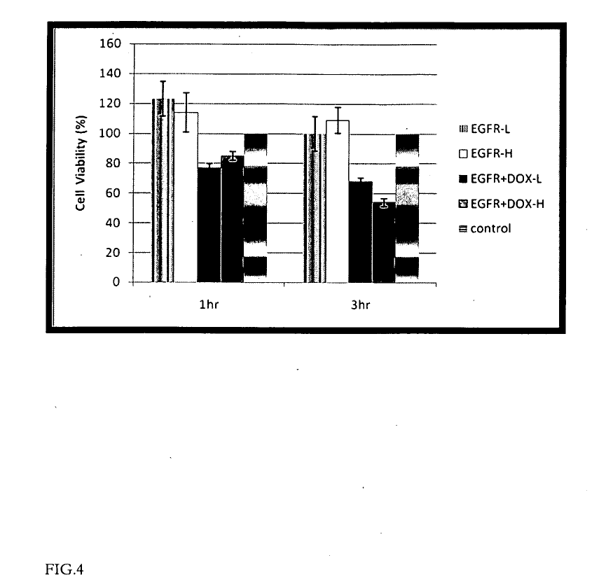

Focusing further on our EGFR-mAb nanocapsules, as proof-of-concept experiment we next tested their ability to encapsulate the chemotherapeutic agent DOX, to which A375 melanoma cells are known to be sensitive. A375 cells were seeded in 96 well plates and treated with low and high dose nanocapsules±DOX which are expected to correspond to 40 and 100% transfection efficiency, based on results shown in FIG. 2. To minimize nonintended effects due to leakage of DOX from the capsules into the media, cells were only incubated with the nanocapsules for 1 or 3 hr before they were removed and incubated in media. After 2 days, cell viability and proliferation was determined by MTS assay. As shown in FIG. 4, absence of DOX has little effect regardless of incubation time. However, when loaded with DOX, a dose—and time—dependent inhibition of proliferation is observed. These results show that transfection of the melanoma cells by the nanocapsules per se does not inhibit proliferation, but that only the encapsulated anticancer agent affects the cancer cell's ability to proliferate. This again we feel is an important characteristic for a therapeutic delivery agent. This will be further investigated in our proposed animal studies.

What is claimed is:

1. A carrier system for administering polyanionic molecules to target cells, which consists of ligand-targeted polycation-coated liposomes formed by incorporating into the liposomal vehicle a biodegradable polycation chemically coupled to a ligand having a high affinity for predefined receptor sites of said target cells, said ligand being capable of acting as a cell targeting agent toward said target cells and as a cell internalization vector.

2. The carrier of claim 1, wherein said biodegradable polycation is selected from the group consisting of a polysaccharide, polyglucosamine, oligoglucosamine, chitosan, a polypeptide, polylysine, polyarginine and copolymers thereof.

3. The carrier of claim 2, wherein the polycation enhances crossing of the target cell membrane.

4. The carrier of claim 1, wherein said polycationic polymer coating is a covalent bonding of a poly(alkylene glycol) and a biodegradable polymer selected from the group consisting of polysaccharides, polyglucosamine, chitosan, a polypeptide, polylysine, polyarginine and copolymers thereof.

5. The carrier of claim 1, wherein said polycationic polymer coating is composed of biodegradable polymer such as chitosan having a molecular weight of between about 100 Dalton and about 20,000 Dalton.

6. The carrier of claim 1, where the biodegradable polymer is comprised of chitosan with a deacetylation of 100%, preferably greater than 90%, most preferably greater than 80%.

7. The carrier of claim 1, wherein the liposome is neutral or anionic and the vesicle forming lipid is selected from the group consisting hydrogenated soy phosphatidylcholine, distearoylphosphatidylcholine, sphingomyelin, diacyl glycerol, phosphatidyl ethanolamine, phosphatidylglycerol, distearylphosphatidylcholine, and distearyl phosphatidylethanolamine.

8. The carrier of claim 1 where the coated liposomes have a diameter less than 2 microns, preferably less than 1 micron, most preferably between 30 and 500 nanometers.

9. The carrier of claim 1, whereas the polycation is coupled to a lysis agent, preferably a pH-sensitive endosomolytic peptide.

10. The carrier of claim 1, wherein said polycation coating is coupled to a biocompatible polyethylene glycol to improve stealth and circulatory properties of the carrier, said polyethylene glycol having a molecular weight less than 10,000 Dalton, preferably less than 5000 Dalton and most preferably less than or equal to 2000 Dalton.

11. The carrier of claim 1, wherein the polycation coating contains an agent which functions to improve the crossing, fusion and uptake of the carrier across the target cell membrane, wherein said agent consists of the D and L forms of polyarginine, nona-D-arginine with or without a spacer, D and L forms of polylysine, polyglucosamine and polyacetylglucosamine.

12. The carrier of claim 11, wherein the spacer between the polycation and the agent is chosen from polyethylene glycol, poly(alkylene glycol) of molecular weight less than 10,000 Dalton, preferably less than 5000 Dalton, most preferably less than or equal to 2000 Dalton.

13. The carrier of claim 1, further comprising a ligand connected to the biodegradable polycationic polymer, said ligand selected from the group consisting of an antigen, a hapten, a vitamin, a protein, a polypeptide, biotin, nucleic acids, DNA, RNA, aptamers, polynucleic acids, a polysaccharide, a carbohydrate, a lectin, a lipid and combination thereof.

14. The carrier of claim 13, wherein the ligand is an antibody, Fab or a fragment thereof.

15. The carrier of claim 13, wherein the ligand binds to a viral antigen, the extracellular domain of signaling membrane proteins such as epidermal growth factor receptor, HER2/neu receptor, basic fibroblast growth factor receptor, vascular endothelial growth factor receptor, tumor necrosis factor receptor, insulin growth factor receptor, folate receptor, cell adhesion molecules such as E-selectin receptor, P-selectin receptor, L-selectin receptor, integrin receptors, chemokine receptors or other growth factor receptors.

16. The carrier of claim 13, wherein the ligand binds to a target cell or tissue specific antigen such as prostate specific membrane antigen, TROY, lymphocyte antigens and tumor antigens.

17. The carrier of claim 16, wherein the target or the antigen on the targeted cell is preferably an internalizable target, less preferably non-internalizable.

18. A method for administering polyanionic molecules to target cells with a carrier system according to claim 1, consisting of ligand-targeted polycation-coated liposomes formed by incorporating into the liposomal vehicle a biodegradable polycation chemically coupled to a ligand having a high affinity for predefined receptor sites of said target cells, said ligand being capable of acting as a cell targeting agent toward said target cells and as a cell internalization vector.

19. A method according to claim 18, wherein said biodegradable polycation is selected from the group consisting of a polysaccharide, polyglucosamine, oligoglucosamine, chitosan, a polypeptide, polylysine, polyarginine and copolymers thereof.

20. A method according to claim 18, for transporting a polyanionic molecule, comprising a therapeutic and/or diagnostic agent across a membrane of a cell by forming a complex between the polyanion molecule and the polycationic coating.

21. A method according to claim 18, wherein the polyanion is selected from the group consisting of RNA, RNAi, siRNA, shRNA, miRNA, small non-coding RNA, aptamers nucleic acids, nucleosides, oligonucleotides, antisense oligonucleotides, DNA.

22. A method of claim 18, wherein the therapeutic agent entrapped in the liposomes is selected from, but not limited to, the group consisting of antibiotics, antivirals, anti-inflammatory, anti-immune agents, antitumor drugs and prodrugs, antibodies, peptides, polypeptides, peptide mimetics, hormones and enzymes.

23. A method of claim 18, wherein the diagnostic agent entrapped in the liposomes is selected from, but not limited to, the group consisting of molecular imaging agents, fluorescent dyes, neutron activation or radiolabeled compounds, lanthanides.

24. A method of claim 18, wherein both the complexed polyanionic macromolecule as well as the entrapped therapeutic and/or diagnostic agent can be delivered simultaneously to a specific target cell.

25. The method of claim 18, further comprising a targeting moiety connected to the biodegradable polycationic polymer selected from the group consisting of a ligand, an antigen, a hapten, a vitamin, a protein, a polypeptide, biotin, nucleic acids, DNA, RNA, aptamers, polynucleic acids, a polysaccharide, a carbohydrate, a lectin, a lipid and combination thereof.

26. The method of claim 18, wherein the ligand is an antibody, Fab or a fragment thereof.

27. The method of claim 18, wherein the ligand binds to an extracellular domain of a membrane protein, a viral antigen, the extracellular domain of signaling membrane proteins such as epidermal growth factor receptor, HER2/neu receptor, basic fibroblast growth factor receptor, vascular endothelial growth factor receptor, tumor necrosis factor receptor, insulin growth factor receptor, folate receptor, E-selectin receptor, P-selectin receptor, L-selectin receptor, integrin receptors chemokine receptors or other growth factor receptors.

28. The method of claim 18, wherein the ligand binds to a target cell or tissue specific antigen such as prostate specific membrane antigen, TROY, lymphocyte antigens and tumor antigens.

29. The method of claim 18, wherein the target or the antigen on the targeted cell is preferably an internalizable target, less preferably non-internalizable.
